(19) United States
(12) Patent Application Publication
(10) Pub. No.: US 2002/0034503 A1
(43) Pub. Date: Mar. 21, 2002

(54) Retrovirus-mediated gene transfer

(76) Inventors: Lorne Taichman, Port Jefferson, NY (US); Soosan Ghazizadeh, Stony Brook, NY (US)

Correspondence Address: Braman & Rogalskyj, LLP, P.O. Box 352, Canandaigua, NY, 14424-0352, US

(21) Appl. No.: 09/964,094

(22) Filed: Sep. 26, 2001

Related U.S. Application Data
(63) Continuation of application No. 09/037,547, filed on Mar. 9, 1998, now abandoned.

(51) Int. Cl. A61K 48/00; C12N 15/86

(52) U.S. Cl. 424/93.21; 435/456

(57) ABSTRACT

The invention provides a method for gene transfer into skin which comprises introducing a foreign gene into a retrovirus, and introducing the retrovirus into dividing skin cells in situ.

This invention was made with support from the United States Government under National Institutes of Health Grant No. RO1 DE04511-14 and No. F32 AR08390.

### FIELD OF THE INVENTION

The subject invention is directed generally to gene transfer, and more particularly to retrovirus-mediated gene transfer into skin in situ.

### BACKGROUND OF THE INVENTION

Throughout this application various publications are referenced, many in parenthesis. Full citations for each of these publications are provided at the end of the Detailed Description. The disclosures of each of these publications in their entireties are hereby incorporated by reference in this application.

Somatic gene therapy involves transfer and expression of a foreign gene in tissues or somatic cells for the purpose of partial or complete replacement therapy (Mulligan 1993; Verma and Somia 1997). Several technical developments have made gene therapy possible. The first is recombinant DNA which allows isolation and rearrangement of genes from various cell types. The second is the development of expression vectors which permit transcription of an encoded gene in mammalian cells. The third is the development of methods and vectors for the transfer of new genetic material to a variety of host cells. The most efficient means of gene transfer entails the use of a crippled virus to introduce the new genetic information into the host cell (transduction). The most studied viral vector utilizes retroviral sequences (Miller et al. 1993). Following entry into cells, retroviruses reverse transcribe their RNA genome and the resulting double stranded DNA intermediate is integrated at high efficiency into cellular DNA (Miller et al. 1993). A retroviral vector genome can accommodate 7-8 kilobases of cloned genes. The recombinant genome can then be encapsidated by suitable helper cell lines (Miller 1990). The resulting replication-defective retrovirus is then an efficient transducer of genes for a wide variety of host cells.

Recent advances in retrovirus vectors for gene therapy can be summarized as follows: (1) The use of newly designed packaging cell lines to reduce or eliminate the likelihood of contamination with replication-competent virus (Danos and Mulligan, 1988): (2) The use of an extended packaging signal to facilitate incorporation of viral genomic RNA into capsids and increase titers (Dranoff et al. 1993); (3) The use of pseudotyping with VSV-G protein to permit concentration of virus stocks (Yee et al. 1994); (4) The incorporation of viral splicing sequences and sequences 5′ to the envelope AUG start site to improve message splicing and message translation (Krall et al. 1996); (5) The use of novel envelope proteins to broaden the virus host range and improve virus attachment and penetration (Yee et al. 1994; Lam et al. 1996; Bayle et al. 1993); (6) The use of vectors targeted to specific receptors (Kasahara et al. 1994; Valsesia-Wittmann et al. 1994); (7) The use of tissue-specific promoters to enhance and prolong expression in the target tissue (Schnierle and Groner, 1996); and (8) The use of lentiviral vectors to target nonreplicating target cells (Naldini et al. 1996a; Naldini et al. 1996b).

Cutaneous gene therapy has several features that make it attractive as an additional approach to gene therapy: (a) A range of culture models is available that allows either for rapid growth and clonal expansion or complete epithelial differentiation (Rheinwald and Green, 1975; Parenteau et al. 1992). These models allow for accurate preclinical assessment of gene-modified cells prior to their use in clinical trials. (b) Considerable experience in skin grafting and grafts of autologous cultured keratinocytes has been used successfully for over a decade to treat severe thermal injuries (Gallico, III et al. 1984). (c) Keratinocyte stem cells have been identified in vivo (Bickenbach and Mackenzie, 1984) and have been recovered for use in culture (Jones and Watt, 1993). (d) A number of keratinocyte-specific promoters have been cloned and characterized and could be incorporated into a retroviral vector (Eckert et al. 1997). (e) Inducible promoters used to express the therapeutic gene might be regulated by topically applied regulators. (f) Virtually all the genetically altered tissue may be monitored in situ and the entire procedure arrested by graft excision if any untoward reaction develops.

Genetically altered epithelia may be useful for treating cutaneous disorders and for systemic delivery of a therapeutically beneficial gene product (for review of the potential applications of keratinocyte gene therapy see (Taichman, 1994; Krueger et al. 1995; Vogel et al. 1996; Khavari and Krueger, 1997)). Studies have shown that for certain genetic disorders of epidermis, introduction of the normal allele for laminin 5 and for transglutaminase I will correct the mutant epidermolysis bullosum and lamellar ichthyosis phenotypes respectively, both in culture as well as in grafts of those cells (Choate et al. 1996b; Choate et al. 1996a). Systemic delivery of a secreted gene product has been demonstrated for several proteins including apolipoprotein E (Fenjves et al. 1994), factor IX (Gerrard et al. 1993), growth hormone (Teumer et al. 1990) and α₁-antitrypsin (Setoguchi et al. 1994).

Gene transfer for therapeutic purposes can be performed ex vivo or in vivo. Ex vivo gene transfer has two key advantages including high efficiency gene transfer and the ability to assess the performance of gene-altered tissue prior to its therapeutic use. However, ex vivo gene transfer for cutaneous gene therapy suffers from one essential disadvantage, namely a likely requirement for full thickness excision of skin at the graft site. If portions of epidermis remain or if portions of hair follicles remain, keratinocytes in these remaining tissues will rapidly grow out and re-epithelialize the denuded surface. This regrowth will undermine the grafts of gene-altered cells and cause graft failure. Full thickness excision at the graft site will result in scarring and contracture, complications that will limit the total surface that will be used for therapy. The only solution to this is to develop an in vivo gene transfer procedure.

U.S. Pat. Nos. 4,868,116 and 4,980,286 also relate to genetically transformed epidermal grafts. In these patents, foreign DNA is introduced into keratinocytes when the keratinocytes are cocultured (ex vivo) with infectious virus (such as retrovirus) in which there is a recombinant genome having foreign genetic material. The keratinocytes can then be grown to confluence, removed as an epithelial sheet, and applied to the body as a graft. These methods for genetically transforming skin rely on in vitro transformation of the skin and then grafting of the transformed skin to a recipient. However, because grafting is a surgical procedure with attendant complications, a need exists for a method for genetically transforming skin in vivo.

### SUMMARY OF THE INVENTION

The subject invention addresses this need by providing a method for gene transfer into skin. The method comprises introducing a foreign gene into a retrovirus, and introducing the retrovirus into dividing skin cells in situ.

### BRIEF DESCRIPTION OF THE DRAWINGS

These and other features and advantages of this invention will be evident from the following detailed description of preferred embodiments when read in conjunction with the accompanying drawings in which:

FIGS. 1A-1D illustrate the re-epithelialization of dermabraded mouse skin;

FIG. 2 illustrates a method of introducing the retrovirus into dividing skin cells by injection of a retrovirus solution between a scab and the dividing skin cells (produced by dermabrasion); and

FIGS. 3A-3D illustrate the results of in vivo transduction of mouse skin.

### DETAILED DESCRIPTION OF THE INVENTION

The subject invention provides a method for gene transfer into skin. The method comprises introducing a foreign gene into a retrovirus, and introducing the retrovirus into dividing skin cells in situ. As used herein, “gene transfer” is intended to include transient gene transfer (temporary) as well as permanent gene transfer, although permanent gene transfer is preferred.

“Skin”, as used herein, refers to the in situ skin of living animals, including humans, mice, and other animals of interest. Gene transfer into humans is useful for gene therapy purposes, and gene transfer into other animals, for example a mouse, can provide useful models for research. A mouse into which a gene has been transferred in accordance with the method of the subject invention can replace a transgenic mouse for research purposes.

The foreign gene can be any nucleic acid of interest, and can be best defined by defining the applications of the subject method to skin disorders. The method of the subject invention can be used for the treatment of inherited or acquired cutaneous disorders (see Alper 1991 and Novice 1994 for discussions of genetic disorders of the skin)(examples of inherited disorders: Junctional epidermolysis bullosum, an autosomal recessive disorder where the lack of one of the functional components of the basement membrane leads to “fragile skin” with blister formation; Xeroderma pigmentosum, an autosomal recessive disorder where there is an inherited lack of one of the components required for repair of ultraviolet light-damaged DNA. Examples of acquired diseases: Chronic nonhealing ulcers, introduction of gene(s) that improve wound healing; Neoplasia, introduction of gene capable of inducing normal differentiation in descendant cells). The method of the subject invention can also be used for the treatment of systemic disorders by delivery of bioactive compounds through production and secretion from gene-modified skin cells (for example: Clotting factor VIII or factor IX for hemophilia A or B; Apolipoprotein E for familial hypercholesterolemia III; Insulin for diabetes mellitus). The method of the subject invention can also be used for the treatment of systemic disorders through a cutaneous intracellular enzyme reservoir capable of metabolically processing circulating substrates (for example: Cutaneous deamination of circulating (deoxy)adenosine for severe combined immunodeficiency caused by the absence of adenosine deaminase; Cutaneous transamination of circulating ornithine to glutamate semialdehyde for gyrate atrophy (blindness disorder) caused by the inherited lack of ornithine aminotransferase). The method of the subject invention can also be used as a diagnostic/research procedure to determine the effect of a specific gene on cutaneous morphology and metabolism. At the present time, the most effective way to determine how expression of a gene in skin alters cutaneous structures and physiology is to construct transgenic mice in which transgene expression is directed at skin. This is a complex, costly and time consuming method. In vivo cutaneous gene transfer with retrovirus vectors offers a relatively simple and less costly method of monitoring the effect of a gene on cutaneous physiology.

The retrovirus is chosen because it will integrate a proviral copy of its vector genome into the skin cell genome upon infection of the skin cells. Integration provides a mechanism for ensuring replication of the newly introduced genetic material and distribution to descendant cells, thereby providing for long term, sustained therapy. Presently preferred retroviruses are pseudotyped retroviruses such as those retroviruses pseudotyped with Vesicular Stomatitis Virus (VSV). Pseudotyped retroviruses are known in the art (see, for example, Sung et al. 1997; Yee et al. 1994; Burns et al. 1993; Reiser et al. 1996; Chen et al. 1996; Goldberg 1992). There are at least three reasons why the vector used in the method of the subject invention is preferably a retrovirus. First, because the retrovirus vector integrates into the genome of the keratinocyte or fibroblast, the transgene will be replicated and inherited by all descendant cells, thus providing for a permanent gene therapy. Second, in all applications, the same “drug” is used, i.e. a retrovirus. Once delivery and safety issues are resolved, development of new applications will be simplified and less costly. Third, if the retrovirus is delivered to a specific cell type or is active only in a specific cell type, then the gene product encoded within that retrovirus vector will not reach other cell types, thus reducing toxicity or other adventitious effects.

The retrovirus is integrated into the genome of dividing cells. Therefore, the skin cells in situ must be dividing. This can be accomplished with the procedure known as dermabrading, for example. The dermabrasion causes a scab to form over the dividing skin cells, and the retrovirus can be introduced between the scab and the dividing skin cells. This introduction can be done by injection of a solution of the retrovirus, using, for example, a needle, between the scab and the dividing skin cells. By injecting the retrovirus between the scab and the dividing skin cells, the scab acts as a barrier to keep the retrovirus in contact with the dividing skin cells.

### EXAMPLE I

### Generation of High Titer Pseudotyped Retrovirus Vectors

For in vivo transduction of epidermal keratinocytes or dermal fibroblasts, high titer retrovirus vectors must be employed. An example of such high titered vectors is the retrovirus vector pseudotyped with VSV-G protein. These pseudotyped viruses are constructed according to published procedures (Yee et al. 1994; Burns et al. 1993). A plasmid containing the retroviral vector and the transgene (e.g. a gene encoding growth factors, receptors, adhesion molecules, enzymes or antisense RNA) is constructed using conventional recombinant DNA technology. Plasmid DNA encoding the retroviral vector is transfected into an amphotropic packaging line such as PA317. Culture supernatant containing infectious virus is collected 48 hrs after transfection and used to transinfect a second packaging line, 293GP cells which stably expresses the Mo-MLV gag and pol genes but do not contain DNA encoding the retroviral envelope protein (Burns et al. 1993). A selectable marker (i.e. neor gene) can be included in the vector to allow positive selection of cells containing the transgene. Colonies are selected in drug-containing medium and the integrity and levels of transgene expression are determined by analysis of cellular DNA and RNA, respectively. The 293GP clone generating the highest transgene activity is identified, expanded and transiently transfected with a plasmid (pHCMV-G) containing vesicular stomatitis virus (VSV)-G glycoprotein using conventional methods of transfection. Expression of the VSV-G encoding plasmid results in formation and release of infectious but replication-incompetent VSV-G pseudotyped virus into the culture medium over a two to four day period. Pseudotyped retrovirus can be concentrated from culture medium by ultracentrifugation without significant loss in virus titer (Yee et al. 1994). Titers of the concentrated stocks are determined by infection of 3T3 cells followed by drug selection. This method allows production of virus stocks with titers as high as 109 CFU/ml.

### EXAMPLE II

### Induction of Hyperplasia Prior to Administration of Retroviruses

There are two principles followed in developing the invented method for delivery of the retrovirus: i) because retrovirus requires cell division for successful transduction, administration is performed following induction of hyperplasia at a time point when mitotic activity is maximal; and ii) because the virus must have access to the progenitor cells in epidermis, barriers to this access, such as the stratum corneum or a hyperplastic suprabasal compartment, must be removed or circumvented to allow access to basal keratinocytes.

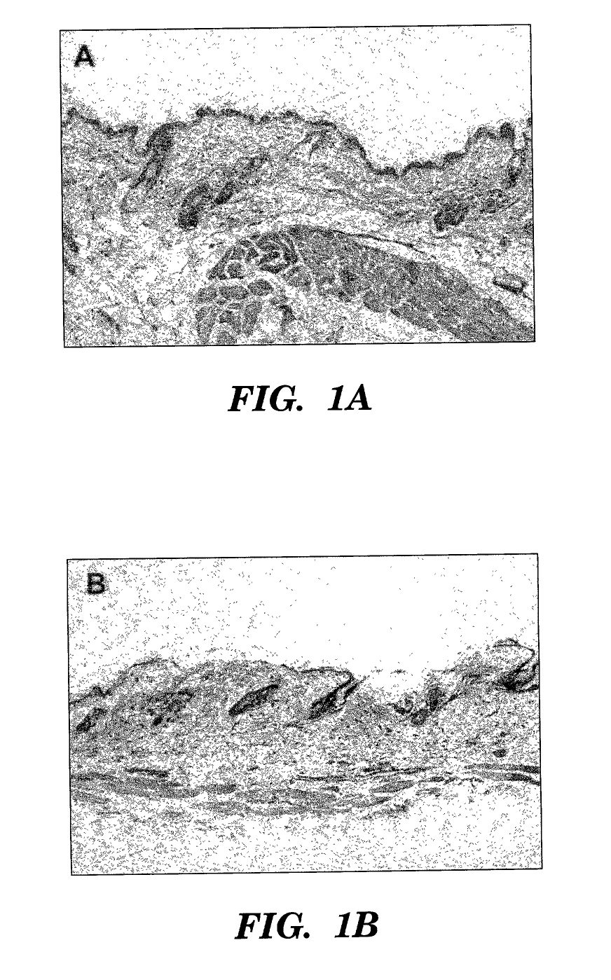

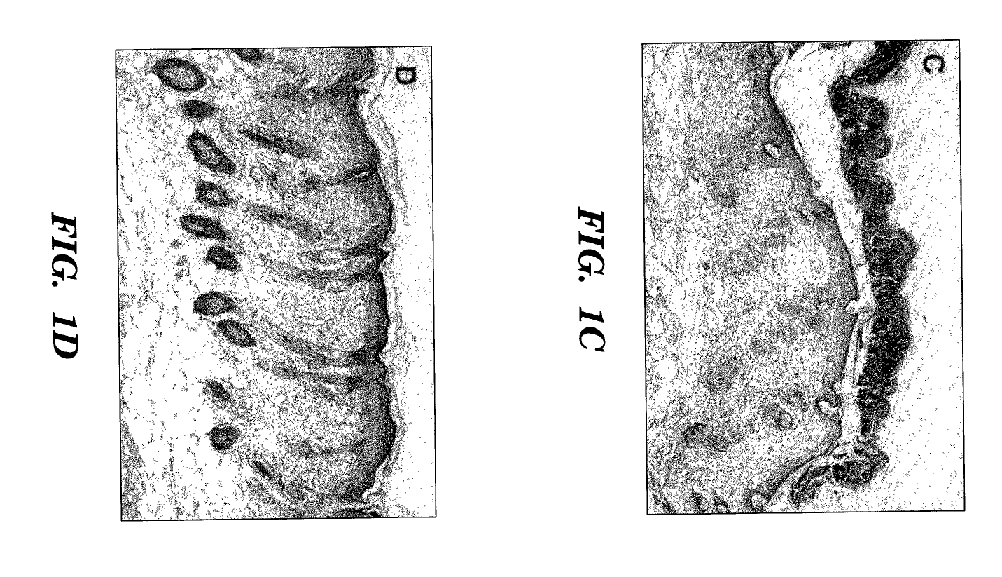

Abrasion of mouse skin with a felt wheel has been shown to remove the epithelial cells resulting in rapid regeneration of epithelium (Argyris 1976). The effect of dermabrasion on normal mouse skin and the rapid regeneration that follows is illustrated in FIGS. 1A-1D. FIG. 1A shows normal mouse skin; FIG. 1B shows mouse skin following dermabrasion, and showing removal of the epidermis; FIG. 1C shown mouse skin 3 days following dermabrasion, and showing a scab and the compartment situated between the scab and the reepithelializing surface; and FIG. 1D shows mouse skin 5 days following dermabrasion.

The following experiment was performed to define the optimum conditions for administration of retroviruses and shows that both aforementioned criteria can be accomplished using dermabrasion. Mice, 7-9 week-old mice, in the resting phase of the hair growth cycle, were anesthetized and the back skin was abraded with felt wheel (⅝″ diameter, ⅛″ thickness) connected to a rotary motor tool spinning at approximately 15,000 rpm. The extent of re-epithelialization was assessed by harvesting the abraded skin at days 0-7 post abrasion. To determine the rate of proliferation (labeling index) in newly formed epidermis, mice were pulse labeled with an intraperitoneal injection of BUdR (50 mg/kg). Three hours later, skin specimens were harvested, fixed and embedded in paraffin and stained either with hematoxylin and eosin or an antibody to BUdR-labeled DNA. The mitotic response to dermabrasion and the histological appearance of the tissue is noted in Table I. These results indicated that the optimal time for inoculation of the retroviruses is day 2-3 post abrasion when the high mitotic activity of keratinocytes and minimal stratification of the re-epithelializing tissue allows the accessibility of the proliferative basal cell population to the viruses.

### EXAMPLE III

### Expression of the Transgene

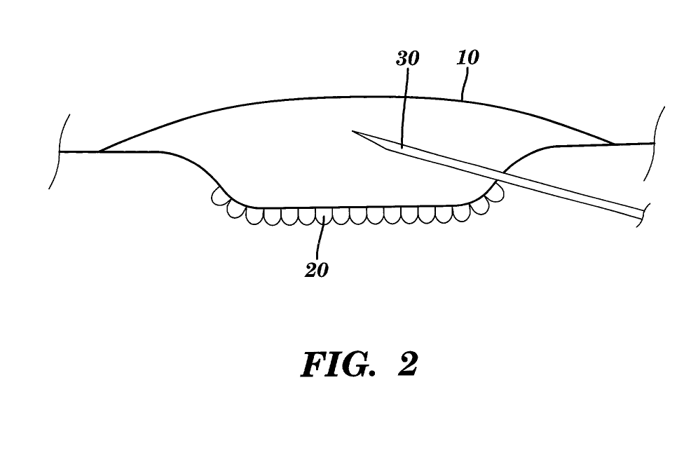

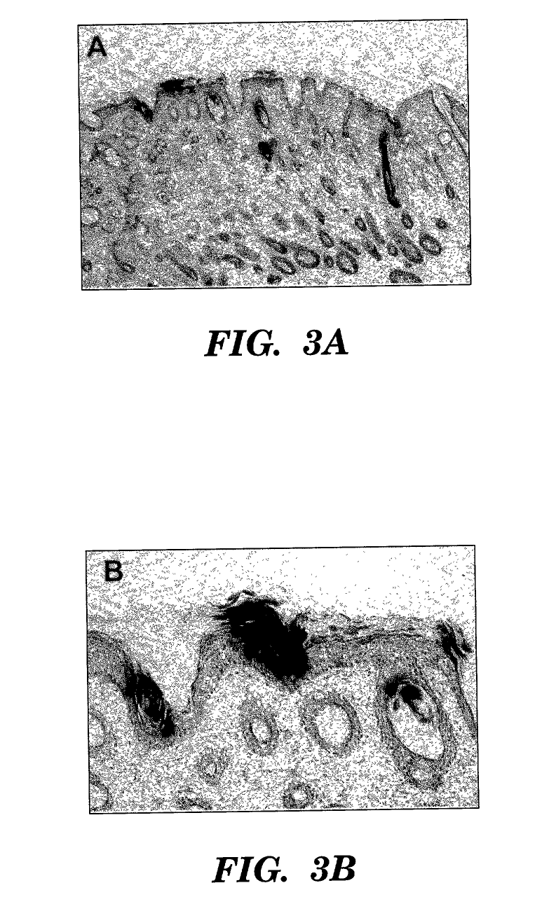

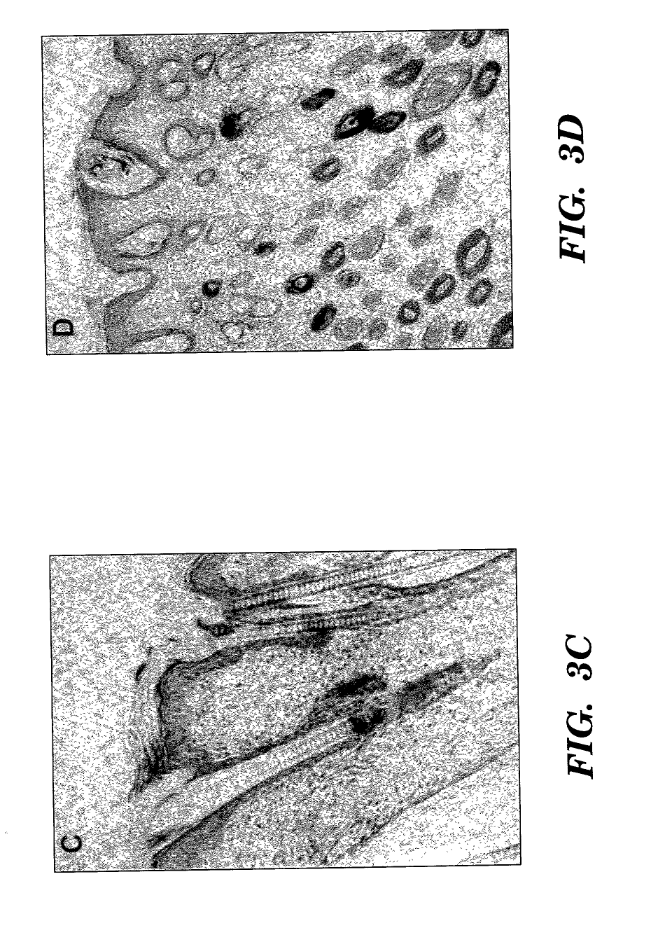

The following experiment was performed to demonstrate the targeting of murine epidermis with a transgene using pseudotyped-retroviruses and to assess transgene expression in epidermal cells. A retrovirus containing E. coli lacZ reporter gene controlled by the viral promoter located in 5′ long terminal repeat of the vector and a selectable marker gene neor controlled by RSV promoter was pseudotyped with VSV-G protein and concentrated and resuspended in Tris-buffered saline (pH 7.6). The concentrated virus was kept at −80° C. until used. The viral titer was 1-2×10⁹ CFU/ml as determined on NIH-3T3 cells. Female Sencar mice (7 week old) were anesthetized and placed on a warm heating pad. The dormal skin was clipped, depilated with Nair™ and an area of about 2 cm² was dermabraded using the technique described in Example II. The wound thus created was allowed to remain open to the air. Three days after dermabrasion, 50 μl (containing 5×10⁷ CFU) of virus was deposited with the aid of a 29 gauge syringe into the compartment located between the scab and the healing tissue surface as shown in FIG. 1C, entering through normal skin 1-2 mm from the edge of the scab (as illustrated in FIG. 2, which shows the scab 10 and the reforming epithelium 20 and the needle 30). A slight lateral force was exerted on the skin prior to inserting the needle so that upon withdrawing the needle no exit track would be present that allows leakage of the virus stock. Twelve days later the expression of lacZ gene in transduced skin was assessed. Skin specimens from abraded areas were biopsied, snap frozen in O.C.T. embedding compound and 10 μm-thick sections were prepared. Sections were fixed for 10 min in paraformaldehyde, washed and stained en face with 4-chromo-5-bromo-3-indolyl-β-D-galactoside (Xgal). Slides were rinsed and counter-stained with eosin/hematoxylin. As shown in FIGS. 3A-3D in specimens taken 12 days post-transduction, specific, intense staining is visible in both follicular and interfollicular epithelium. In interfollicular epidermis, distinct columns of Xgal positive cells are apparent spanning basal, spinous and granular cells. Staining is also apparent in sebaceous glands and in various portions of the hair follicle.

Transduced cells can be positively selected by topical application of an appropriate selective drug (for example, Geneticin or G418 for vectors carrying the gene encoding neomycin phosphotransferase). This allows selective expansion of transduced cells in the area exposed to the drug, since the drug is toxic to cells that do not harbor the foreign gene (for example, the neomycin phosphotransferase)

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

TABLE I

| Days post | Labeling index | Presence of | Epidermal |
|---|---|---|---|
| abrasion | (%) * | scab | layers ** |
| normal skin | 5 | − | 2-3 + SC |
| 0 | — | − | 0 |
| 1 | 5 | − | 0-1 |
| 2 | 21 | + | 1-2 |
| 3 | 25 | + | 2-3 + SC |
| 4 | 30 | + | 4-6 + SC |
| 5 | 60 | − | 7-8 + SC |
| 7 | 56 | − | 7-8 + SC |

* The labeling index was determined by injecting the animals with bromodeoxyuridine (BUdR) and 6 hours later harvesting tissue from the dermabraded area. BUdR-positive cells were detected in paraffin-embedded histological sections using an antibody to BUdP-labeled DNA (Boehringer-Mannheim, Inc). The labeling index is the percentage of BUdR-positive cells in the basal compartment.
** The number of layers comprising the basal, spinous and granular layers is indicated by the numbers. The SC indicates the presence of a stratum corneum.

### References

Alper, J. C., Genetic Disorders of the Skin, Mosby Year Book, St. Louis (1991).

Argyris, T. S., Am J Pathol 83:329-340 (1976).

Bayle, J. Y., et al., Hum Gene Ther 4:161-170 (1993).

Bickenbach, J. R. and I. C. Mackenzie, J Invest Dermatol 82:618-622 (1984).

Burns, J. C., et al., Proc Natl Acad Sci USA 90:8033-8037 (1993).

Chen, S. T., et al., Proc Natl Acad Sci USA 93:10057-10062 (1996).

Choate, K. A., et al., Hum Gene Ther 7:2247-2253 (1996a).

Choate, K. A., et al., Nature (Medicine) 2:1263-1267 (1996b).

Danos, O. and R. C. Mulligan, Proc Natl Acad Sci USA 85:6460-6464 (1988).

Dranoff, G., et al., Proc Natl Acad Sci USA 90:3539-3543 (1993).

Eckert, R. L., et al., J Invest Dermatol 109:501-509 (1997).

Fenjves, E. S., et al., Hum Gene Ther 5:1241-1248 (1994).

Gallico, G. G., III, et al., N Engl J Med 311:448-451 (1984).

Gerrard, A. J., et al., Nature (Genetics) 3:180-183 (1993).

Goldberg, D. M., Clin Chim Acta 206:45-76 (1992).

Jones, P.H. and F.M. Watt, Cell 73:713-724 (1993).

Kasahara, N., et al., Science 266:1373-1376 (1994).

Khavari, P. A. and G. G. Krueger, Adv. in clinical research 15:27-35 (1997).

Krall, W. J., et al., Gene Ther 3:37-48 (1996).

Krueger, G. G., et al., J Invest Dermatol 103:76S-84S (1995).

Lam, J. S., et al., Hum Gene Ther 7:1415-1422 (1996).

Miller, A. D., Hum Gene Ther 1:5-14 (1990).

Miller, A. D., et al., Meth Enzymol 217:581-599 (1993).

Mulligan, R. C., Science 260:926-932 (1993).

Naldini, L., et al., Proc Natl Acad Sci USA 93:11382-11388 (1996a).

Naldini, L., et al., Science 272:263-267 (1996b).

Novice, F. M., Handbook of Genetic Skin Disorders, W. B. Saunders, Philadelphia (1994).

Parenteau, N. L., et al., Cytotechnol 9:163-171 (1992).

Reiser, J., et al., Proc Natl Acad Sci USA 93:15266-15271 (1996).

Rheinwald, J. G. and H. Green, Cell 6:331-344 (1975).

Schnierle, B. S. and B. Groner, Gene Ther 3:1069-1073 (1996).

Setoguchi, Y., et al., J Invest Dermatol 102:415-421 (1994).

Sung A. D., et al., J Virol 71:1397-1404 (1997).

Taichman, L. B., in: I. M. Leigh, E. B. Lane and F. M. Watt (Eds.), The Keratinocyte Handbook, Cambridge University Press, Cambridge, pp. 543-551 (1994).

Teumer, J., et al., FASEB J 4:3245-3250 (1990).

Valsesia-Wittmann, S., et al., J Virol 68:4609-4619 (1994).

Verma, I. M. and N. Somia, Nature 389:239-242 (1997).

Vogel, J. C., et al., Adv Dermatol 11:383-398 (1996).

Yee, J. K., et al., Proc Natl Acad Sci USA 91:9564-9568 (1994).

What is claimed is:

1. A method for gene transfer into skin comprising:
introducing a foreign gene into a retrovirus; and
introducing the retrovirus into dividing skin cells in situ.

2. The method of claim 1 wherein the retrovirus is a pseudotyped retrovirus.

3. The method of claim 2 wherein the pseudotyped retrovirus is pseudotyped with Vesicular Stomatitis Virus.

4. The method of claim 1 wherein the dividing skin cells are induced into division by dermabrasion.

5. The method of claim 4 wherein the dermabrasion causes a scab over the dividing skin cells, and wherein the retrovirus is introduced between the scab and the dividing skin cells.

6. The method of claim 5 wherein the retrovirus is introduced by injection as a solution of retrovirus.

7. The method of claim 1 further comprising applying a selective drug to the skin to allow expansion of skin cells having the retrovirus introduced therein.
